(19) United States
(12) Patent Application Publication
(10) Pub. No.: US 2014/0094686 A1
(43) Pub. Date: Apr. 3, 2014

(54) MEDICAL INSTRUMENT, MEDICINE SYSTEM AND METHOD FOR DISPLAYING THE POSITION OF A POSITION MARKER

(71) Applicant: SIEMENS AKTIENGESELLSCHAFT, Muenchen (DE)

(72) Inventor: NASSIR NAVAB, MUENCHEN (DE)

(73) Assignee: SIEMENS AKTIENGESELLSCHAFT, Muenchen (DE)

(21) Appl. No.: 14/042,987

(22) Filed: Oct. 1, 2013

(30) Foreign Application Priority Data
Oct. 1, 2012 (DE) 102012217942.9

(51) Int. Cl. A61B 5/06

(52) U.S. Cl. CPC A61B 5/066; A61B 5/065
USPC 600/424

(57) ABSTRACT

A medical instrument contains at least three navigation markers which in each case have at least one marker element which can be captured by a camera in a camera image in an identifiable manner. The navigation markers are each attached to the instrument at an attachment point lying on a first straight line. At least one of the navigation markers contains at least three marker elements arranged at a known distance on a second straight line associated with the respective navigation marker. Moreover, a medicine system and a method display a position of a position marker spatially correlated with the medical instrument.

### CROSS-REFERENCE TO RELATED APPLICATION

This application claims the priority, under 35 U.S.C. §119, of German application DE 10 2012 217 942.9, filed Oct. 1, 2012; the prior application is herewith incorporated by reference in its entirety.

### BACKGROUND OF THE INVENTION

### Field of the Invention

The invention relates to a medical instrument, a medicine system and a method for displaying the position of a position marker spatially correlated with a medical instrument.

By way of example, surgical interventions are carried out on patients as medical measures. By way of example, this also includes the treatment of fractures of bones, into which implants are inserted. Here, the implants have to be inserted and affixed to the individual bone fragments to be treated in such a way that these can heal in a desired relative position with respect to one another. Here, fixing the implant to the individual bone fragments is a procedure in which different medical instruments, such as interlocking screws or medical tools such as drills are employed. In order to establish the demanded relative position of the individual bone fragments, the medical instruments such as interlocking screws or drills employed during the treatment have to be positioned in the correct manner. However, this is complicated by the fact that target positions for instruments lie in the body interior of the patient and are therefore not visible to the eye. By way of example, the target position for an interlocking screw is a hole within an implant, which in turn is situated within the bone.

Conventionally, the position of a medical instrument is monitored by an x-ray image. Then, the current position of the instrument and the target position, for example the hole of the implant, can be seen in such an x-ray image. There can then be a correction on the basis of the deviation between the current position and the target position. However, a disadvantage in such a method is that both patient and medical staff are exposed to a high x-ray radiation dose.

However, in order to reduce the radiation load, there are also methods by which so-called navigation-assisted surgery is carried out. To this end, navigation markers with marker elements which can be captured by optical cameras in an identifiable manner are attached to the involved medical instruments. Prior to medical measure, the respective marker elements have to be calibrated with the navigation system containing the optical camera. A current position of an instrument can then be established on the basis of the images of the marker elements identified in the camera image. This current position can then in turn be compared to a target position, whereupon correction measures are displayed to the medical staff in order, for example to reach a target position. However, a disadvantage in this case is that optical navigation markers can be covered by objects in the treatment region or by the medical staff themselves, and so identification of the images of the marker elements, and hence navigation, is prevented.

### BRIEF SUMMARY OF THE INVENTION

It is therefore an object of the present invention to specify a medical instrument, a medicine system and a method for displaying the position of a position marker spatially correlated with the medical instrument, by which the aforementioned disadvantages are avoided.

The object mentioned first is achieved by a medical instrument containing at least three navigation markers which in each case have at least one marker element which can be captured by a camera in a camera image in an identifiable manner. The individual navigation markers are each attached to the instrument at an attachment point lying on a first straight line. At least one of the navigation markers contains at least three marker elements arranged at a known distance on a second straight line associated with the respective navigation marker.

By virtue of the fact that at least one navigation marker contains at least three marker elements, it is possible with the aid of the known distances of the individual marker elements on the second straight line to establish mathematically the position of the attachment point of the navigation marker on the medical instrument which likewise lies on the second straight line. Thus, it is not necessary to attach a marker element directly on the instrument. The marker elements therefore have a specific distance from the instrument and are therefore not covered, for example when held by the hands of the medical staff, when they are handled during a medical measure. In the extreme case, all necessary navigation markers are configured such that the marker elements are not situated directly on the instrument itself, but rather are in each case arranged at a specific distance on a second straight line which extends away from the attachment point of the instrument. In order to obtain a good ability to identify the individual images of the marker elements in a camera image, the second straight lines extend in different directions.

If the positions of the at least three attachment points lying on a first straight line are then established in a camera coordinate system and if the distances of the attachment points of the instrument are known, it is also possible to establish the positions of further points which are characteristic to the instrument and lie on this first straight line. Here, these can for example be points of an instrument which cannot be seen by the eye during a medical measure, for example because they are covered by the patient body. Hence a target-oriented navigation of the instrument is possible, even without the use of x-ray radiation.

In order to improve further the handling of the instrument, the navigation marker containing marker elements arranged on a second straight line is attached to the instrument rotatable about the attachment point on the first straight line. Hence the instrument can be gripped by the medical staff and the navigation marker can, even during a medical measure, be rotated into such a position that the marker is not covered in respect of the recording by a camera. As a result, an identification of the images of the marker elements and a navigation of the instrument are always ensured.

In order to detect the position of one end of an instrument in a simple manner, a navigation marker is formed by an individual marker element attached to one end of the instrument. Hence, the marker element forming the navigation marker is attached directly to the attachment point and so the position of the attachment point of the navigation marker and of the marker element correspond. In this case, it is possible, in the process, to dispense with a complicated embodiment of the navigation marker by several marker elements lying on a second straight line. The ability to identify a marker element in the camera image is usually ensured in the case where the marker element attached to the distal end of an instrument in particular.

In a preferred embodiment of the invention, a medical instrument has exactly three navigation markers, wherein two navigation markers in each case contain exactly three marker elements arranged at a defined distance on a second straight line. Thus, as described above, the positions of two of the attachment points can, in the case of such an instrument, initially be established mathematically in a camera coordinate system, whereas the position of the attachment point at which the navigation marker formed by a marker element is established directly on the basis of the position of the image thereof in a camera image. Hence the positions of the attachment points lying on the first straight line are known. On the basis of these positions and the known actual distances it is then possible, in turn, to establish further characteristic points on this first straight line. Here, this can for example be the tip of an interlocking screw or else of a drill. Hence, in order to establish the position of the tip in the camera coordinate system, the tip itself need not be equipped with a marker element. Hence, the position of this point spatially correlated with the instrument is established indirectly by establishing the positions of the individual marker elements.

In a preferred embodiment of the invention, the first straight line coincides with a central longitudinal axis of the instrument, as, for example, is expedient in the case of a screw or a drill.

For simple detection of the individual marker elements by a camera, the surface of the marker elements has a fluorescent or light-reflecting design.

So as to be able to distinguish the marker elements better, these can have different shapes, such as, for example, the shape of a sphere or a pyramid. In particular, the marker elements associated with one navigation marker can have the same shape, but the marker elements associated with a different navigation marker can have a shape differing therefrom.

The object mentioned second is achieved by a medicine system containing: a medical instrument according to the invention, a camera which generates a camera image, and a computer unit which establishes the position of the marker elements in the camera coordinate system on the basis of the position of the images of the marker elements in the camera image.

In accordance with a preferred embodiment of the invention, the medicine system additionally has an x-ray device which generates an x-ray image and is preferably arranged on a C-arm, in which the camera is integrated into the x-ray device in such a way that the viewing direction thereof coincides with the imaging direction of the x-ray device.

As a result, it is easily possible to superpose x-ray images and camera images at the correct position and additionally to superimpose position information in respect of the medical instrument.

The object mentioned third is achieved by a method for displaying the position of a position marker spatially correlated with a medical instrument.

Accordingly, a camera image containing the images of the marker elements is created by a camera in step a).

In step b), the positions of the images of the marker elements in the camera image are established by a computer unit.

In step c), the position of the marker elements in the camera coordinate system is established by the computer unit on the basis of the position of the images of the marker elements in the camera image.

In step d), the position of the position marker spatially correlated with the instrument is established in the camera coordinate system on the basis of known geometric relationships and displayed on a user interface.

The current position of a marker element or navigation marker can be displayed by the position marker spatially correlated with the instrument. However, it is also possible to display the position of a point which is established mathematically on the basis of the positions of the marker elements using known geometric relationships, such as, for example, the position of an attachment point of a navigation marker on the instrument. Hence, it is also possible to display the current position of the tip of an interlocking screw by the position marker. It is therefore also possible to display positions of points on an instrument which cannot be seen by eye or in the camera image since these, for example, are covered by the body of the patient. However, the method according to the invention enables a real-time display of the current position, even during a medical measure without using x-ray radiation.

In a preferred embodiment of the method, the position marker is superimposed into the camera image at the correct position in step d). The medical staff therefore obtains the information which current position therefore has a point spatially correlated with the instrument in the camera image.

In a further preferred embodiment of the method, an x-ray image of a treatment region is generated prior to step a). The position marker is then superimposed into the x-ray image at the correct position in step d) on the basis of known geometric relationships of the camera coordinate system and x-ray coordinate system. Hence the position information in respect of the position marker can also be provided in respect of the x-ray image. Overall, it is also possible to obtain a combination of x-ray image, camera image and position marker.

In order to support the navigation, a target marker for the position marker can be additionally superimposed into the x-ray image. Accordingly, the medical staff obtains both the specification of the position marker, i.e. the current position of the instrument, and the desired target position thereof. Hence the staff is able to assess in which direction the instrument has to be moved in order to bring it into the target position.

The properties, features and advantages of this invention described above, and also the manner in which these are achieved will become clearer and more understandable in conjunction with the following description of the exemplary embodiments, which are explained in more detail in conjunction with the drawings.

Other features which are considered as characteristic for the invention are set forth in the appended claims.

Although the invention is illustrated and described herein as embodied in a medical instrument, a medicine system and a method for displaying the position of a position marker, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a perspective view of a medical instrument according to the invention;

FIG. 2 is an illustration of a medicine system according to the invention;

FIG. 3 is an illustration of an x-ray image;

FIG. 4 is an illustration of a camera image;

FIG. 5 is an illustration of a user interface with a displayed position marker; and

FIG. 6 is an illustration of a user interface with a superposed x-ray image and camera image as well as a position marker.

### DESCRIPTION OF THE INVENTION

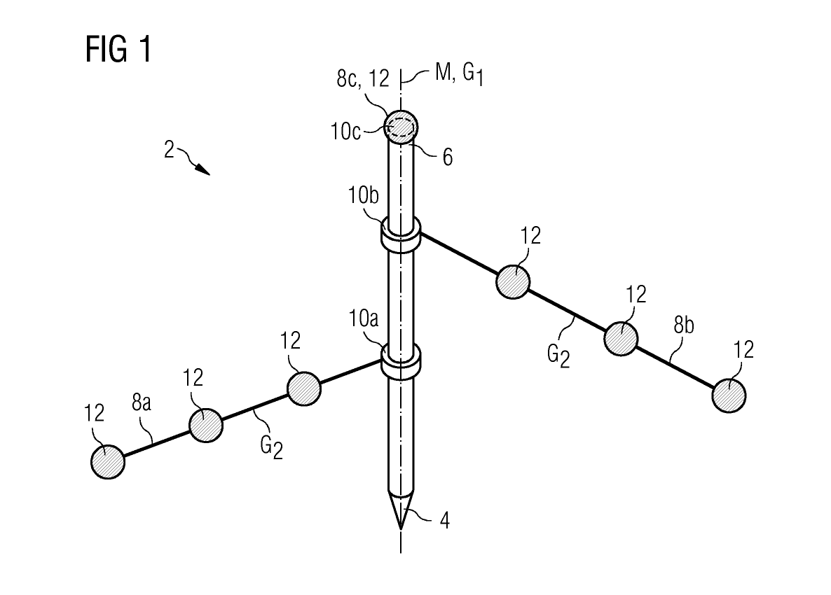

Referring now to the figures of the drawing in detail and first, particularly, to FIG. 1 thereof, there is shown a medical instrument 2 according to the invention, which is a Schanz screw in this exemplary embodiment. The medical instrument 2 has a central longitudinal axis M and a tip 4 and an end 6. Attached to the medical instrument 2 are three navigation markers 8a, 8b, 8c, respectively at an attachment point 10a, 10b, 10c. The attachment points 10a, 10b, 10c lie on a first straight line G₁, which coincides with the central longitudinal axis M of the medical instrument 2. The distances of the individual different attachment points 10a, 10b, 10c on the straight line G₁ are known. The navigation marker 8c is formed by an individual marker element 12, which can be detected in a camera image in an identifiable manner. Hence the marker element 12 associated with the navigation marker 8c is attached directly to the attachment point 10c, and so the position of the marker element 12 and of the attachment point 10c coincide.

The navigation markers 8a, 8b each have exactly three marker elements 12 which are each arranged at a known distance on a second straight line G₂ associated with the respective navigation marker 8a, 8b. Hence, overall, the instrument 2 has seven marker elements 12. The navigation markers 8a, 8b are attached to the instrument 2 in a manner rotatable about the respective attachment points 10a, 10b on the first straight line G₁. In order to enable good detectability of the marker elements 12 in a camera image, the surfaces of the marker elements 12 have a fluorescent or light-reflecting design. In the exemplary embodiment, the individual marker elements have a spherical shape. In order to ensure that the individual marker elements 12 can be distinguished better, the diameter of the marker element 12 respectively associated with a navigation marker 8a, 8b, 8c differs. By way of example, the marker elements 12 associated with the navigation marker 8a have a diameter of 1 cm, the marker elements associated with the navigation marker 8b have a diameter of 1.2 cm and the marker element associated with the navigation marker 8c has a diameter of 2 cm. However, it is also possible to ensure that the individual marker element 12 can be distinguished by virtue of the fact that the marker elements 12 have a different shape such as e.g. the shape of a sphere or a pyramid.

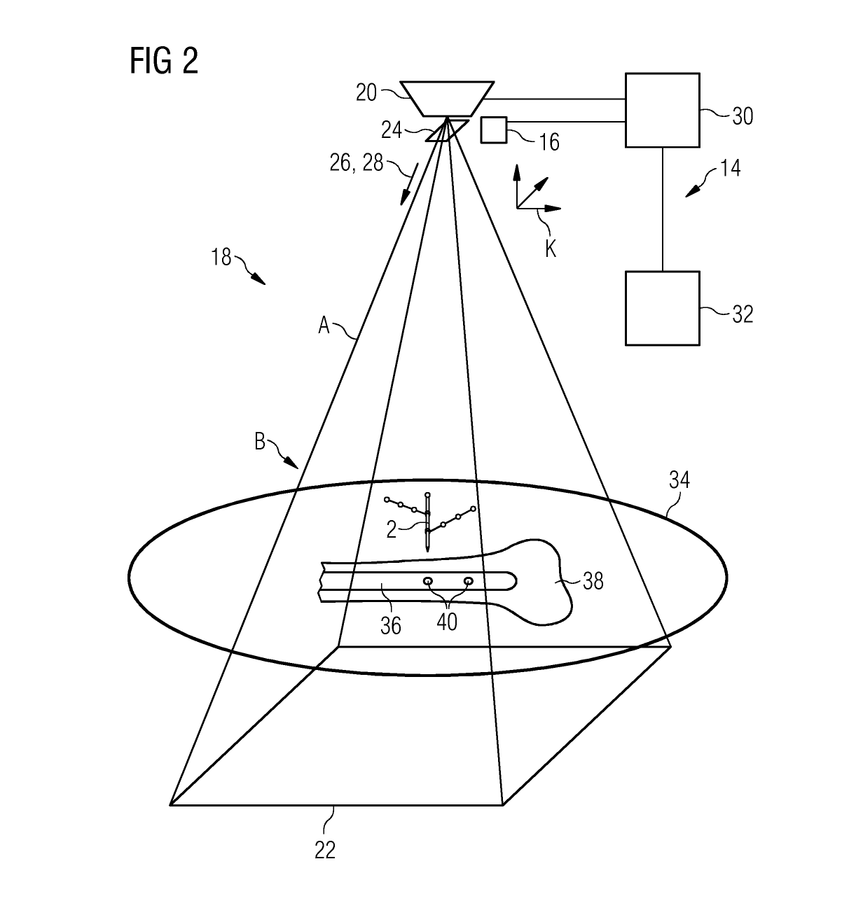

FIG. 2 now depicts a medicine system according to the invention which initially contains the medical instrument 2 according to the invention. Moreover, a medicine system 14 contains a camera 16 which generates a camera image 46 and an x-ray device 18, which contains an x-ray source 20 and an x-ray detector 22. By way of example, the x-ray source 20 and the x-ray detector 22 are affixed to a C-arm (not depicted). In order to have the same recording geometry A as the x-ray device, a mirror 24 is arranged on the camera 16. Hence, the camera 16 is integrated into the x-ray device 18 such that a viewing direction 26 thereof coincides with a imaging direction 28 of the x-ray device 18.

Moreover, the medicine system 14 contains a computer unit 30, which is connected to the x-ray device 18 and to the camera 16 and can establish the position of the marker elements 12 in a camera coordinate system K on the basis of the positions of the images of the marker elements 12 in the camera image. A user interface 32 such as, for example, a monitor for outputting information for the medical staff is in turn connected to the computer unit 30.

FIG. 2 also simultaneously depicts an initial situation for carrying out the method according to the invention. Within the scope of an operation, an implant 36 serving to treat a bone 38 in the exemplary embodiment was introduced into a body 34 of a patient. In order to affix the implant 36 on the bone 38, the former has several holes 40, through which suitable attachment devices have to be introduced. In order to mark the point of use on the bone 38, a Schanz screw representing the medical instrument 2 must initially be brought or navigated with the aid of the invention into a position over the hole 40.

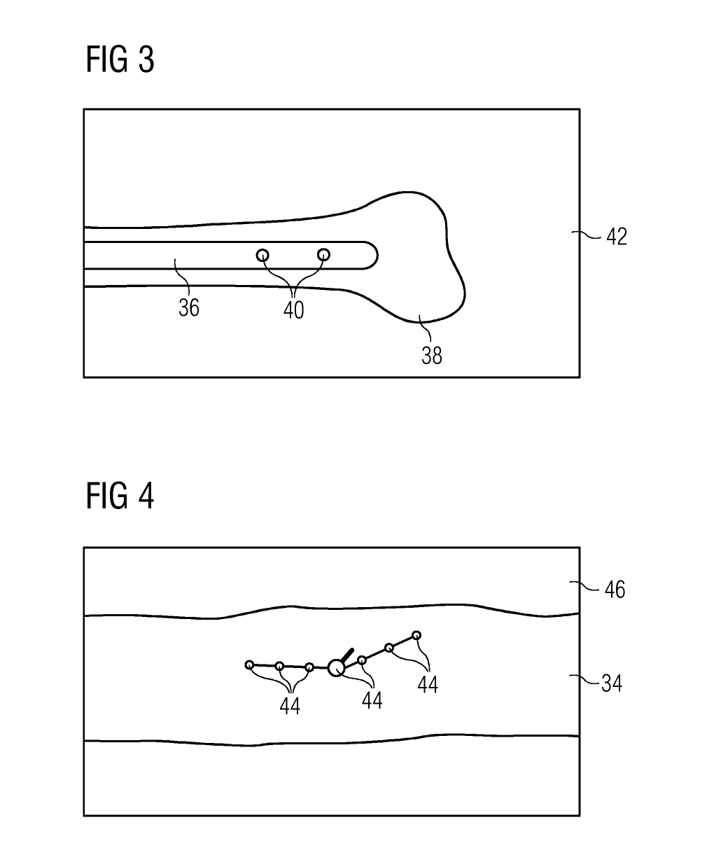

In order to ensure this, an x-ray image 42 is initially created of the treatment region B; this is displayed in FIG. 3. In it, it is possible to identify the bone 38 and the implant with holes 40 introduced therein.

In step a) of the method according to the invention, the camera 16 is used to create a camera image 46 which contains a treatment region B and hence also images 44 of the marker elements 12; the camera image is depicted in FIG. 4. It is furthermore possible to see the surface of the body 34 of the patient in this camera image 46, but not the implant 36 and the holes 40 thereof, since it is covered by the bone 38.

The positions of the images 44 of the marker elements 12 in the camera image 46 are established in step b) by the computer unit 30.

The position of the marker elements 12 in the camera coordinate system K is established in step c) by the computer unit using the positions of the images 44 of the marker elements 12 in the camera image 46.

In step d), the position of the position marker 48, spatially correlated with the instrument 2, is established in the camera coordinate system K and displayed on the user interface 32 on the basis of known geometric relationships. Here, the position marker 48 shows the current position in the camera coordinate system K of a point which has a fixed geometric relation to the marker elements 12, i.e. it is spatially correlated thereto. The position marker 48 could therefore represent the current position of the marker element 12 itself but also that of a point which is spatially correlated to the marker elements 12.

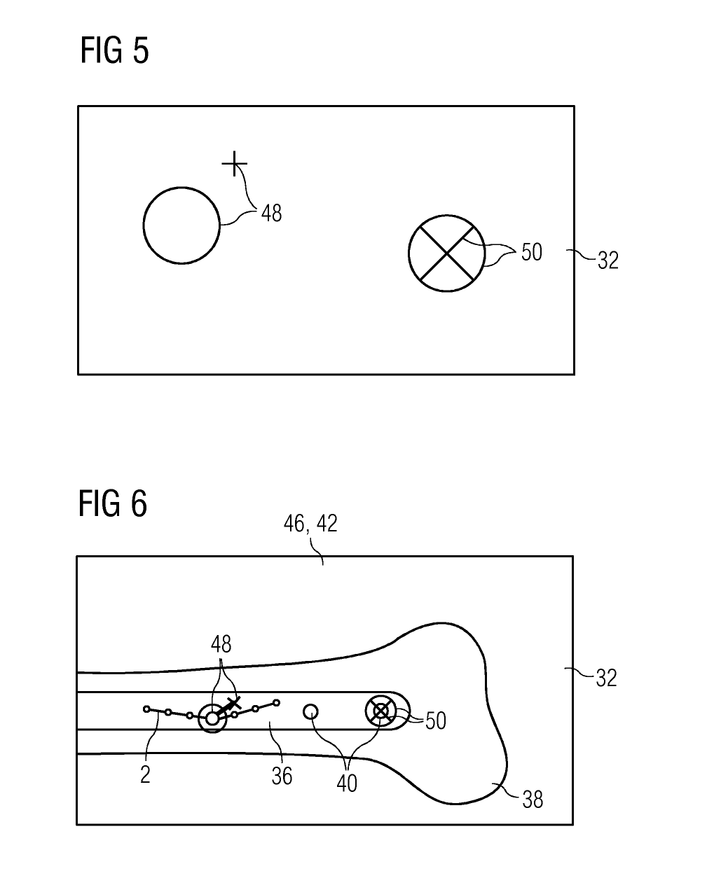

In this case, a position marker 48, which is depicted in FIG. 5 and FIG. 6 as a plus sign, represents the position of the tip 4 of the instrument 2. This therefore means that the current position of the tip 4 of the instrument 2 is displayed on the user interface 32. A user can therefore see the position of the tip 4 on the user interface 32, even though the tip 4 for example is already situated in the body 34 of the patient and hence is no longer visible with the naked eye.

In order to calculate the position of the tip 4 of the medical instrument 2 in the camera coordinate system K, the positions of the marker elements 12, established in step c), are used and the positions of the attachment points 10a, 10b, 10c lying on the straight line G₁ and of the tip 4 are initially established. The position of the attachment point 10c here emerges directly from the position of the marker element 12 associated with the navigation marker 8c.

By contrast, the positions of the attachment points 8a, 8b must be established mathematically.

If the positions of three points A, B, C lying on a straight line are known in the camera coordinate system, it is also possible to calculate the position of a fourth point D in the camera coordinate system provided that the actual distances of the points are known.

In this respect, the following relationships apply, where dᵢⱼ are the distances of the points ij in the camera coordinate system K, i.e. of the marker elements 12:

$$cross ratio = \frac{AB \cdot CD}{AC \cdot BD}$$

$${d}_{AD} = \frac{( S \cdot {d}_{AB} ) - {d}_{AC}}{S - 1}$$

$$S = \frac{cross ratio \cdot {d}_{AC}}{{d}_{AB}}$$

Thus, using these relationships, it is possible to establish the positions of the attachment points 8a, 8b using the positions of the images 44 of the marker elements 12, established in step c), since the attachment points 8a, 8b respectively constitute a fourth point on the respective straight line G₂, wherein the positions of three points on the straight line G₂, namely those of the marker elements 12, are known.

After establishing the positions of the three attachment points 10a, 10b, 10c, the positions of three points on one straight line, namely the straight line G₁, are known in turn. Thereupon, it is possible in turn to use the aforementioned relationships to establish the position of a fourth point on this straight line G₁, i.e. in this case the position of the tip 4 of the instrument 2. This position is then displayed on the user interface using the position marker 48.

In this case, a further position marker 48 is displayed as a circle using the user interface 32, which further position marker displays the position of the marker element 12 associated with the navigation marker 8c and hence the position of the end 6 of the medical instrument 2.

Furthermore, the user interface 32 displayed the target position for the tip 4, namely the position on the bone 38 above the hole 40, by a target marker 50 configured as an X and the target position for the end 6 of the instrument 2 by the target marker 50 configured as a circle. Hence the user interface 32 provides the medical staff with information relating to the direction in which the tip 4 or the end 6 of the instrument 2 needs to be moved. If the respective position markers and target markers 50 are congruent, the instrument 2 is situated at the desired target position.

It is now possible to see in FIG. 6 that both the camera image 46 and the x-ray image 42 are displayed in a superposed manner by the user interface. Hence the bone 38 and the implant 36 with the holes 40 can be seen in the composed image. Furthermore, the medical instrument 2 is also contained in the image of the user interface 32. Moreover, both the spatially correlated position marker 48 of the tip 4 and of the end 6 of the medical instrument 2 is superimposed into the image. Additionally, the target markers 50 for the tip 4 and also the end 6 of the medical instrument above the one hole 40 are also displayed.

This renders it possible for the medical staff to be able to move the medical instrument 2 into the required target position without this requiring separate x-ray monitoring.

Although the invention was illustrated and described in more detail by the preferred exemplary embodiment, the invention is not limited by the disclosed examples and a person skilled in the art is able to derive other variations from this, without departing from the scope of protection of the invention.

1. A medical instrument, comprising:
at least three navigation markers in each case having at least one marker element which can be captured by a camera in a camera image in an identifiable manner; and
a medical instrument body, said navigation markers each attached to said medical instrument body at an attachment point lying on a first straight line and at least one of said navigation markers having at least three marker elements disposed at a known distance on a second straight line associated with said at least one navigation marker.

2. The medical instrument according to claim 1, wherein said at least one navigation marker containing said marker elements disposed on the second straight line is attached to said medical instrument body rotatable about said attachment point on said first straight line.

3. The medical instrument according to claim 1, wherein one of said navigation markers is formed by an individual one said marker element attached to one end of said medical instrument body.

4. The medical instrument according to claim 1, wherein said navigation markers are exactly three navigation markers, wherein two of said navigation markers in each case contain exactly three said marker elements disposed at a known distance on the second straight line in each case.

5. The medical instrument according to claim 1, wherein the first straight line coincides with a central longitudinal axis of said medical instrument body.

6. The medical instrument according to claim 1, wherein said marker elements have a surface with a fluorescent design.

7. The medical instrument according to claim 1, wherein said marker elements have a surface with a light-reflecting design.

8. The medical instrument according to claim 1, wherein said marker elements have a differing shape.

9. The medical instrument according to claim 1, wherein said marker elements have a spherical design.

10. A medicine system, comprising:
a camera for generating a camera image;
a medical instrument containing at least three navigation markers in each case having at least one marker element which can be captured by said camera in the camera image in an identifiable manner, said medical instrument further having a medical instrument body, said navigation markers each attached to said medical instrument body at an attachment point lying on a first straight line and at least one of said navigation markers having at least three marker elements disposed at a known distance on a second straight line associated with said at least one navigation marker; and
a computer unit for establishing a position of said marker elements in a camera coordinate system on a basis of a position of images of said marker elements in the camera image.

11. The medicine system according to claim 10, further comprising an x-ray device for generating an x-ray image, said camera integrated into said x-ray device such that a viewing direction of said camera coincides with an imaging direction of said x-ray device.

12. The medicine system according to claim 11, wherein said x-ray device is a C-arm x-ray device.

13. A method for displaying a position of a position marker spatially correlated with a medical instrument containing at least three navigation markers in each case having at least one marker element which can be captured by a camera in a camera image in an identifiable manner, the medical instrument further having a medical instrument body, the navigation markers each attached to the medical instrument body at an attachment point lying on a first straight line and at least one of the navigation markers having at least three marker elements disposed at a known distance on a second straight line associated with the at least one navigation marker, which comprises the steps of:
a) creating a camera image containing images of the marker elements via the camera;
b) determining positions of the images of the marker elements in the camera image by means of a computer unit;
c) establishing a position of the marker elements in a camera coordinate system via the computer unit on a basis of the positions of the images of the marker elements in the camera image; and
d) establishing a position of a position marker spatially correlated with the medical instrument in the camera coordinate system on a basis of known geometric relationships and displayed on a user interface.

14. The method according to claim 13, which further comprises superimposing the position marker into the camera image at a correct position in the step d).

15. The method according to claim 14, which further comprises:
generating an x-ray image of a treatment region prior to the step a); and
superimposing the position marker into the x-ray image at the correct position in step d) on the basis of the known geometric relationships of the camera coordinate system and an x-ray coordinate system.

16. The method according to claim 15, which further comprises superimposing a target marker for the position marker into the x-ray image.
